(19) United States
(12) Patent Application Publication
(10) Pub. No.: US 2019/0376984 A1
(43) Pub. Date: Dec. 12, 2019

(54) METHODS FOR QUANTIFYING SOLUBLE AMYLOID BETA AND AMYLOID BETA OLIGOMERS

(71) Applicants: Daniel Kidd, Pacifica, CA (US); Janice Nickel, Pacifica, CA (US)

(72) Inventors: Daniel Kidd, Pacifica, CA (US); Janice Nickel, Pacifica, CA (US)

(21) Appl. No.: 16/436,244

(22) Filed: Jun. 10, 2019

Related U.S. Application Data
(60) Provisional application No. 62/682,418, filed on Jun. 8, 2018.

(51) Int. Cl. G01N 33/68; A61K 38/17; G01N 33/52; C07K 16/18

(52) U.S. Cl. CPC G01N 33/6896; A61K 38/1716; G01N 33/52; C07K 2317/34; G01N 2800/52; G01N 2800/2821; C07K 16/18

(57) ABSTRACT

Detection, diagnosis, and monitoring of Alzheimer's disease can be achieved with antibody-based detection methods directed to detect at least two regions of the amyloid beta peptide. The invention recognizes and relies on the observation that the steric availability of the carboxyl terminal epitope and the amino terminal epitope in an amyloid beta peptide differs based on whether the amyloid beta is monomeric or oligomeric. Due to oligomerization, the measured concentration of carboxyl termini that are available for binding represents the amount of monomeric amyloid beta in the sample; whereas the measured concentration of amino termini that are available for binding represents total amyloid beta (i.e., monomeric plus oligomeric) in the sample. Comparing the relative binding therefore can be used to characterize the population of soluble amyloid beta oligomers in the sample. The measurements are useful for early detection and subsequent monitoring of the development of Alzheimer's disease in a patient.

### CROSS-REFERENCE TO RELATED APPLICATION(S)

This application claims the benefit of and priority to U.S. Provisional Application Ser. No. 62/682,418, filed Jun. 8, 2018, the contents of which are incorporated by reference in their entirety.

### FIELD OF THE INVENTION

The disclosure generally relates to methods for characterizing and determining quantities of total soluble amyloid beta (Aβ), monomeric amyloid beta and neurotoxic population of amyloid beta oligomers up to and including amyloid beta-derived diffusible ligands (ADDLS) in a sample, data processing of results generate a measure of current neurodegeneration risk which untreated, may lead to Alzheimer's disease.

### BACKGROUND

Alzheimer's disease is an irreversible, progressive degeneration of brain cells associated with deterioration of memory that hampers the affected person's day to day life. Symptoms develop slowly and worsen over time, and the condition is quite debilitating and uniformly fatal. Alzheimer's disease is characterized by plaques and tangles appearing neural tissue. Plaques are deposits of a protein fragment called amyloid beta that build up in the spaces between nerve cells. Tangles are twisted fibers of another protein called Tau that builds up inside cells. Amyloid beta is made when amyloid precursor protein is cleaved by proteases to secrete amyloid beta peptides.

Alzheimer's disease has an extensive preclinical stage, which is initiated 15 to 20 years prior to the emergence of clinical signs. Unfortunately, there is no consensus on a biomarker that reliably predicts Alzheimer's disease during early, pre-clinical stages. Biomarkers such as levels of amyloid beta in cerebrospinal fluid or Tau and P-Tau levels in tissue samples have been proposed. See e.g., U.S. Pat. No. 7,700,309, incorporated by reference. However, researchers have failed to establish correlations between amyloid beta and/or Tau or P-Tau and Alzheimer's disease diagnosis or progression. Additionally, recent failures of potential disease-modifying drugs for Alzheimer's disease may reflect the fact that the enrolled participants in clinical trials are already too advanced to derive a clinical benefit.

### SUMMARY

The disclosure uses the insight that presence and exposure of neural tissue to oligomeric forms of amyloid beta peptide mediate neurodegeneration, loss of mental function and cognitive impairment. Methods for determining what proportion of amyloid beta is present in oligomeric form versus monomeric form is a mechanism for the early quantitation of neurotoxic oligomeric amyloid beta, independent of cognitive status. Amyloid beta oligomers exert their toxicity through a variety of mechanisms including receptor and direct membrane interactions. Amyloid beta peptides form oligomers via interactions among their carboxyl-termini. Accordingly, methods of the disclosure include measuring amyloid beta peptides in a sample, and also measuring a quantity of the carboxyl-terminus of the amyloid beta peptides that are available within the sample for binding. If substantially all of the amyloid beta peptides present also have their carboxyl-termini available for binding, the amyloid beta is inferred to be present as soluble monomers, which is taken to be negative or low neurodegeneration risk. A measure of amyloid beta present in a sample accompanied by a determination that relatively fewer or no carboxyl-termini are available for binding indicates that the amyloid beta peptides are present predominantly in oligomeric forms, thus the method serves as a marker of pre-clinical Alzheimer's disease.

From early on-set in preclinical stages detection, diagnosis, and monitoring of Alzheimer's disease is achieved with antibody-based detection methods directed to detect at least two regions of the amyloid beta peptide. The invention recognizes and relies on the observation that in an amyloid beta peptide the steric availability of the carboxyl-terminal epitope and the amino-terminal epitope differs based on whether the amyloid beta is monomeric or oligomeric. Subject risk of neurodegeneration is proportional to oligomeric amyloid beta present in cerebrospinal fluid (CSF). The duration of exposure increases cumulative neurodegeneration. The methods described yield a results continuum with normal subjects of low risk at one extreme to higher at risk subjects, based on oligomeric amyloid beta characterization algorithms. The methods described can identify and quantify the presence of neurotoxic amyloid beta oligomers independent of cognitive assessment, amyloid plaque or metabolic rate imaging, or other biomarkers. Carboxyl-terminal concentration is representative of the amount of monomeric amyloid beta, whereas amino-terminal concentration is representative of the total amyloid beta (i.e., monomeric plus oligomeric). A comparison of the relative antibody binding characterizes the amount and proportion of soluble amyloid beta oligomers in the sample, useful for quantifying current risk of neuronal toxicity, applicable to longitudinal comparisons of treatment effects in clinical studies, or disease progression, or in population observational studies, or as a risk screening, from the preclinical phase to more advanced stages of the disease.

The claimed invention uses antibodies to detect multiple regions of the amyloid beta peptide, providing a proportional internal comparison of amyloid beta to itself to generate a difference or ratio indicative of the extent to which the amyloid beta peptides in the sample have formed oligomers. The sample is exposed to carboxyl-terminal specific immunoreagents and amino-terminal specific immunoreagents. Amyloid beta oligomer structure at the hydrophobic carboxyl-terminal end sterically limits immunoreagent binding. However, the hydrophilic amino-terminus of amyloid beta is not sterically inhibited in soluble amyloid beta oligomers. Since the carboxyl-terminal epitopes in oligomers are sterically unavailable compared to the carboxyl-terminal epitopes in monomeric amyloid beta, the measured carboxyl-terminal concentration is representative of the amount of monomeric amyloid beta. Since the amino-terminal epitopes are sterically available in both monomeric and oligomeric amyloid beta, the amino-terminal concentration is representative of the total amyloid beta (i.e., monomeric plus oligomeric). The comparison of the relative antibody binding can be used to characterize the population of soluble amyloid beta oligomers in the sample. The amount of oligomeric amyloid beta can be deduced from the comparison between carboxyl-terminal binding and amino-terminal binding, and may be represented as a difference between the two, a ratio, or an amount. The invention allows the amount of soluble oligomeric amyloid beta in the sample to be determined and generates a risk statistic report, enabling early detection and early treatment to prevent, delay or modify the course of amyloid mediated Alzheimer's disease.

The invention provides methods of characterizing the populations of amyloid beta monomers and soluble oligomeric amyloid beta in body fluid samples. Such methods include: (a) measuring an amount of amyloid beta carboxyl-terminus, representing monomeric amyloid; (b) measuring an amount of amyloid beta amino-terminus, representing total amyloid (both monomer and soluble amyloid oligomers); (c) comparing the amounts of monomeric amyloid beta and total amyloid beta; and (d) using the comparison to quantify soluble diffusible neurotoxic amyloid beta present in the subject sample.

The comparison may involve determining a ratio, quotient, difference, or a combination thereof. In some embodiments, for example, the comparison involves determining a ratio between the monomeric amyloid beta and the total amyloid beta. A lower quotient of monomeric amyloid beta to total amyloid beta indicates a correspondingly higher degree of soluble amyloid, and is therefore indicative of a greater neurodegeneration risk, greater likelihood of the presence of degenerative disease or deteriorating condition of the subject. In other embodiments the comparison may instead determine a difference between the total amyloid beta and the monomeric amyloid beta. The difference is itself representative of the amount of oligomeric amyloid. A greater difference indicates a greater neurodegeneration risk, greater likelihood of the presence of degenerative disease, or a deteriorating condition of the subject. In still other embodiments, the comparison may determine a ratio between monomeric amyloid beta and oligomeric amyloid beta, a lower quotient of monomeric amyloid beta over oligomeric amyloid beta providing an indication of greater neurodegeneration risk, greater likelihood of presence of degenerative disease or deteriorating condition of the subject. Some methods determine an amount of oligomeric amyloid beta, a higher amount of oligomeric amyloid beta providing an indication of greater neurodegeneration risk, greater likelihood of presence of degenerative disease or deteriorating condition of the subject.

Aspects of the invention involve methods of characterizing amyloid beta. The methods include first measuring a quantity of amyloid beta in a sample and second measuring a quantity of a carboxyl terminal portion of the amyloid beta available for binding in the sample. The methods further involve comparing the quantity of the carboxyl terminal available for binding to the quantity of amyloid beta. Using this comparison, an amount of oligomeric amyloid beta in the sample is quantified. The sample may be blood, plasma, serum, cerebrospinal fluid, or another bodily sample.

In certain embodiments, measuring the quantity of amyloid beta includes contacting the sample with a first antibody that binds to an amino-terminal region of an amyloid beta peptide, and second measuring the quantity of the carboxyl-terminal portion of amyloid beta available for binding includes contacting the sample with a second antibody that binds to the carboxyl terminal.

In some embodiments, the method further involves providing a report that includes an amount of oligomeric amyloid beta in the sample, wherein the amount of oligomeric amyloid beta is the measured quantity of amyloid beta less the determined amount of monomeric amyloid beta. Examples are illustrative, other algorithms are possible alone or in combination with other cognitive assessments, imaging or other biomarkers.

In embodiments, the method involves quantifying a detectable signal from a biomolecular interaction between the amyloid beta peptide and the monoclonal antibodies. The detectable signal may be generated by an assay such as but not limited to the list consisting of: Enzyme linked immunosorbent assay (ELISA), enzyme immunoassays (EIA), immunoblot assays, Western blot assays, immunoprecipitation, enzyme linked immunospot, antibody microarray assays, and/or immunohistochemistry assays (IHC), fluoroimmunoassays (FIA), fluorescent bead based immunoassay, flow cytometric assays, fluorescence-activated cell sorting (FACS), radioimmuno assays (RIA), immunocytochemical (ICH) assays, chemiluminescent assays, electrochemilumenescence (ECL) assays, chromatographic assays either by immunoaffinity or post column signal generation.

The amount of amyloid beta carboxyl terminus corresponds to an amount of monomeric amyloid beta in the sample, while the amount of amyloid beta amino terminus corresponds to an amount of total amyloid beta in the sample. Total amyloid beta includes both monomeric amyloid beta and oligomeric amyloid beta. Quantifying amyloid beta in the sample may therefore involve determining a ratio between the amount of monomeric amyloid beta and total amyloid beta in the sample. From that, the amount of oligomeric amyloid beta in the sample can be deduced by determining a difference between the amount of monomeric amyloid beta and total amyloid beta in the sample.

Monoclonal antibodies are known in the art. The first monoclonal antibody may specifically bind to at least one of Aβ37, Aβ38, Aβ39, Aβ40, Aβ41, or Aβ42. The first monoclonal antibody can be but not limited to commercial reagents 2G3 and MABN11 for anti-amyloid beta x-40. Alternatively, commercial reagents MABN12, MABN13 and 05-831-1 have anti-amyloid beta x-42 specificity. The second monoclonal antibody may specifically bind to Aβ1, Aβ2, Aβ3, Aβ4, Aβ5, Aβ6, Aβ7, Aβ8, Aβ9, Aβ10, or Aβ11. The second monoclonal antibody can be commercial reagents AHP1252, MABN639, 1E8 from BioRad, Millipore and Sigma, respectively. In certain embodiments, one or both of the monoclonal antibodies is a reporter antibody, which can be labeled with but not limited to an electrochemilumenescent or chemiluminescent tag (e.g. ruthenium), an enzyme (e.g. Alkaline phosphastase or Horse radish peroxidase), an affinity tag (e.g. biotin, polyHis), protein ligand or antibody Fc specificity, a fluorescent tag (e.g. fluorescent beads, fluorescein, phycoerythrin, CY3, CY5, or CY9), a radioisotope (e.g. ³H), or other ligands. In certain embodiments, one or both of the monoclonal antibodies is a capture antibody, which can be through Fc specificity, small molecule ligand (e.g. biotin), large molecule ligand (e.g. streptavidin), or bound or covalently attached to a surface or microsphere.

Methods of the invention may further involve assessing a subject's susceptibility to developing Alzheimer's disease based on the quantified amount of soluble diffusible neurotoxic amyloid beta in the sample. The method may also involve determining a stage of Alzheimer's disease or monitoring a response to treatment regimen. In some embodiments, the assessment is used to recommend a treatment regimen.

In a related aspect, the invention includes a kit for characterizing amyloid beta in a sample. The kit includes an antibody specific for a carboxyl terminal epitope of amyloid beta, an antibody specific for an amino terminal epitope of amyloid beta, and an antibody specific for a central epitope of amyloid beta. Kit reporter immunoreagents are dependent on immunoassay technique e.g. ECL uses Ruthenylated reporter, ELISA uses enzyme conjugate reporter, Scintillation counter use radiolabeled reporter, fluorescence detection uses fluorophore reporter conjugates. The carboxyl terminal antibody may be end-specific for Aβ37, Aβ38, Aβ39, Aβ40, Aβ41, or Aβ42. The amino terminal antibody is end-specific for Aβ1, Aβ2, Aβ3, Aβ4, Aβ5, Aβ6, Aβ7, Aβ8, Aβ9 or Aβ10. The kit can be used to perform the methods described herein.

### BRIEF DESCRIPTION OF THE DRAWINGS

FIG. 1 shows a diagram of the processing of amyloid beta precursor protein by β-secretase and γ-secretase to generate and secrete amyloid beta.

FIG. 2 shows a schematic diagram of the binding sites on the amyloid beta peptide.

FIG. 3 shows a computer system for use with the invention.

FIG. 4 shows a process flow for a general approach for a quantification assay.

FIG. 5 shows a process flow for another quantification assay.

### DETAILED DESCRIPTION

The present invention provides methods to measure amyloid beta (Aβ) protein concentrations in a subject sample to determine the relative amounts of monomeric amyloid beta and oligomeric amyloid beta in a sample. Soluble neurotoxic oligomeric amyloid beta present in cerebrospinal fluid represents risk of neurodegeneration. The preclinical characterization of soluble amyloid beta oligomers provides a current risk statistic of amyloid beta multimers, and thereby warns of potential future Alzheimer's disease. These methods are quantified using antibody-based detection in biological fluids such as cerebrospinal fluid or plasma. These methods, alone or in combination with known molecular imaging and neuropsychological testing, achieve the diagnostic sensitivity and specificity necessary to identify people in the earliest stages of Alzheimer's disease when drug modification is most likely possible. The methods can be used in later stages of the disease as well, for diagnosing or monitoring the disease or clinical therapeutic effect or to determine progress over the course of a treatment or to recommend a therapeutic regimen. The method can be used for preclinical screening of potential therapeutics.

Methods of the invention take advantage of the progressive cerebral deposition of the amyloid beta protein in brain regions serving memory and cognition, an invariant and defining feature of Alzheimer's disease. Since these deposits precede cognitive symptoms by years or even decades, treatment and prophylaxis of Alzheimer's disease is facilitated by the disclosed assays, which detect the formation of amyloid aggregates and/or other disease-associated physiological abnormalities prior to the onset of neurodegeneration and cognitive symptoms. The disclosed methods are able to quantify and assign a value to soluble amyloid beta oligomers through direct measurement using antibody-based detection, thereby providing earlier detection and better treatment outcomes than previously available.

The disclosure provides methods for characterizing soluble amyloid beta populations in biological fluids utilizing immunoreagents to establish and quantify peptide concentrations. Concentrations are determined based immunoreagent detection of both the amino terminus (representing total amyloid) and the free carboxylic acid terminus (representing amyloid beta monomers) using the same reporter molecule to enable comparison of concentration results. The assays are performed in duplicate at one or more dilutions with acceptance criteria for accuracy and precision within dilution replicates. Among carboxyl terminal monomer determinations, the calculated neat concentration estimates from lower dilutions may be higher in samples with soluble amyloid beta multimers, which at lower concentrations dissociate to a new equilibrium increasing amyloid beta monomer. Such results demonstrate the presence of soluble amyloid beta multimers.

As used herein, the term “antibody” includes intact antibodies and binding fragments thereof. Typically, fragments compete with the intact antibody from which they were derived for specific binding to an antigen. Fragments include separate heavy chains, light chains, Fab, Fab′, F(ab)′2, Fabc, scFv, diabodies, Dabs, and nanobodies. Fragments are produced by recombinant DNA techniques, or by enzymatic or chemical separation of intact antibodies.

The disclosed methods involve antibodies that bind specifically to regions of the amyloid beta peptide. “Specific binding” refers to the binding of an antibody (or other reagent) to a target (e.g. a component of a sample) that is detectably higher in magnitude and distinguishable from non-specific binding occurring to at least one unrelated target. Specific binding can be the result of formation of bonds between particular functional groups or particular spatial fit (e.g. lock and key type) whereas nonspecific binding is usually the result of van der Waals forces. Specific binding does not however imply that an agent binds on and only one target. Thus, an agent can and often does show specific binding of different strengths to several different targets and only nonspecific binding to other targets. Specific binding usually involves an association constant of 10⁷, 10⁸, 10⁹ M⁻¹ or higher.

The sites to which the antibodies bind are referred to herein as epitopes. The term “epitope” refers to a site on an antigen to which an immunoglobulin (or antibody or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes includes, for example x-ray crystallography and 2-dimensional nuclear magnetic resonance.

The antibodies described herein comprise residues that bind an epitope. The region containing these residues is known as an “epitope binding region”. When an antibody is said to bind to an epitope within specified residues, such as Aβ1-11, what is meant is that the antibody specifically binds to a polypeptide containing the specified residues (i.e. Aβ1-11 in this example). Such an antibody does not necessarily contact every residue within the Aβ1-11. Nor does every single amino acid substitution or deletion within Aβ1-11 necessarily significantly affect binding affinity.

Some antibodies that will be described herein bind specifically to epitopes at the end of a peptide chain. Such an antibody is referred to as an “end specific antibody” because specifically binds to an epitope at the very N- or C-terminus of an amyloid beta peptide (i.e. the epitope includes the N-terminal or C-terminal amino acid of the peptide) but binds less strongly or does not specifically bind to the residues constituting the epitope in a longer form of amyloid beta or in amyloid beta precursor protein. Thus, an antibody that is end-specific for Aβ40 means that the antibody preferentially binds (e.g. association constant at least ten-fold higher) Aβ40 relative to an amyloid beta peptide ending at residue 37, 38, 39, 41, 42, or 43. Likewise, antibody that is end-specific for Aβ42 means that the antibody preferentially binds an AP peptide ending at residue 42 over an AP peptide ending at residue 37, 38, 39, 40, 41, or 43.

Amyloid beta is formed by the amyloidogenic pathway. Amyloid beta-mediated neurodegeneration of the brain is a leading pathway to Alzheimer's disease. There are two pathways for processing amyloid precursor protein (AβPP): an amyloidogenic pathway and a non-amyloidogenic, constitutive secretory pathway. The Alzheimer's-associated AβPP is cleaved by α-, β-, and presenilin (PS)/γ-secretases through sequential regulated proteolysis, to generate and secrete amyloid as monomeric peptides into the cerebrospinal fluid. The length of amyloid beta (Aβ) varies from 39-43 amino acids. The predominant form, Aβ1-40 is 40 amino acids in length and is considered a short form. The next most common form, Aβ1-42 is 42 amino acids in length and is considered a long form. Aβ1-42 is associated with pathogenicity and is the primary constituent in neuritic plaques (90%) and parenchymal vessel deposits (75%).

The bulk of amyloid beta secreted into cerebrospinal fluid moves across the blood brain barrier and is degraded. A fraction of amyloid beta in the cerebrospinal fluid may form dimers and multimers. Aβ1-42 is a self-associating peptide forming neurotoxins made up of small diffusible amyloid beta oligomers (referred to as amyloid beta-derived diffusible ligands, or ADDLs), which were found to kill mature neurons in organotypic central nervous system cultures at nanomolar concentrations. Further aggregation generates protofibrils, and then fibrils eventually forming non-soluble deposits of amyloid plaque neuropile. The carboxyl terminus of amyloid beta contains hydrophobic residues, which may account for its tendency to aggregate into the fibrils that form plaque.

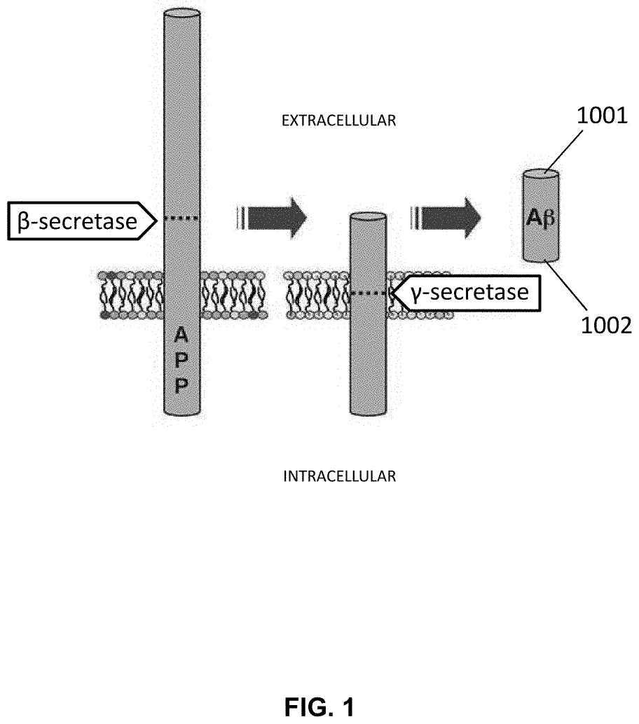

FIG. 1 shows a general diagram of the processing of AβPP by β-secretase and γ-secretase to generate and secrete amyloid beta, which can be used by the present invention. Enzymatic cleavage of the AβPP by β-secretase yields a hydrophilic amino terminus. Enzymatic cleavage by a second, intramembrane site by γ-secretase yields a hydrophobic residue at the carboxyl terminus to generate/secrete amyloid beta. The cleavages of β-secretase and γ-secretase cleavages combine to generate amyloid beta. Aβ1-42 monomers may bind to other amyloid beta peptides. The expected mechanism of amyloid beta binding is through hydrophobic carboxyl terminus interaction to form dimer or higher soluble multiples. Constitutive secretion of amyloid beta monomers can “push” the monomer-multimer equilibrium toward non-soluble aggregates and plaque deposits in the brain. Amyloid beta plaque deposits likewise “pull” the equilibrium through removal of soluble multimers. In time, plaque deposits can be detected in neuro-imaging techniques, although later in the neurodegenerative progression. Methods of the invention take advantage of these interactions by measuring amounts of amyloid beta peptides in a sample such that the extent of oligomerization of the amyloid beta peptides can be determined. According to the present disclosure, the amyloid beta peptide is detected at antibody binding site 1001 and antibody binding site 1002, as shown in FIG. 1.

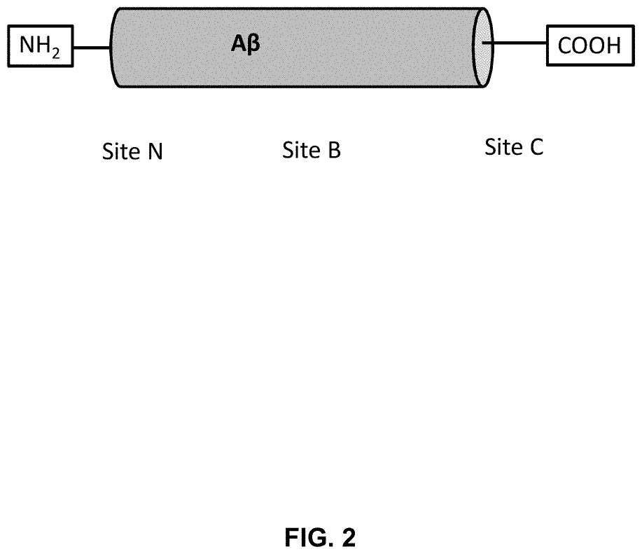

A schematic diagram of the amyloid beta peptide is shown in FIG. 2. The amyloid beta peptide has multiple antibody binding sites, which are useful with the present invention, and which will be described herein. As indicated in the figure, Site N is the region of amino terminus immunoreagent(s) binding; Site C is the region of carboxyl terminus immunoreagent(s) binding; and Site B is the central portion of amyloid beta immunoreagent binding.

The specificity of monoclonal antibodies requires binding partner availability. Some or all of the amyloid beta dimer, trimer, and higher multimer of amyloid beta aggregates are sterically not available to carboxyl terminal specific immunoreagents. Thus when oligomers are present, a proportionately lower signal will be generated from carboxyl terminal specific immunoreagents due to multimer competition. Aβx-42 represents monomeric amyloid population. Antibody that pairs to the amino region (Site N) and the central region (Site B) measures amino terminus concentration. Separately, antibody that pairs to the central region (Site B) and carboxyl region (Site C) determines the carboxyl terminus concentrations.

An algorithm combining results of the binding availability of each terminus can yield a risk statistic. The ratio of carboxyl to amino binding concentrations, for example, can reveal the extent of monomeric versus total amyloid. When this statistic has a value of approximately 1.0, that indicates amyloid beta in the sample is predominately monomeric. A lower value reflects a corresponding reduced signal representing increased amyloid beta aggregates that inhibit binding of carboxyl terminus specific antibodies, likely through steric hindrance. Because this can be detected in patients well before signs of Alzheimer's disease are observed, this antibody-based quantification allows early characterization of subject risk of neurodegeneration due to soluble amyloid aggregates.

Various embodiments of this general concept are envisioned for characterizing the populations of amyloid beta monomers and soluble oligomeric amyloid beta in body fluid samples. In a preferred embodiment, a method involves: (a) measuring an amount of amyloid beta carboxyl terminus representing monomeric amyloid; (b) measuring an amount of amyloid beta amino terminus representing total amyloid (i.e., monomer and soluble amyloid oligomers); (c) comparing the measured amounts of monomeric amyloid beta and total amyloid beta; and (d) using the comparison to quantify a risk statistic related to soluble diffusible neurotoxic amyloid beta in the subject sample. In general, the values of the measured total amyloid beta peptides (Site N) and the carboxyl terminus available for binding (Site C) concentrations will be approximately equal if all amyloid beta peptide is monomeric in the sample, but will be unequal if an amount of amyloid beta is oligomeric.

Some methods determine a ratio between the monomeric amyloid beta and the total amyloid beta, where a lower quotient of monomeric amyloid beta over total amyloid beta provides an indication of greater neurodegeneration risk, greater likelihood of presence of degenerative disease, or deteriorating condition of the subject.

Some methods determine a difference between total amyloid beta and monomeric amyloid beta representing oligomeric amyloid, where a greater difference provides an indication of greater neurodegeneration risk, greater likelihood of presence of degenerative disease, or deteriorating condition of the subject.

Some methods determine a ratio between monomeric amyloid beta and oligomeric amyloid beta, where a lower quotient of monomeric amyloid beta over oligomeric amyloid beta provides an indication of greater neurodegeneration risk, greater likelihood of presence of degenerative disease, or deteriorating condition of the subject.

Some methods determine a ratio between total amyloid beta minus monomeric amyloid beta and total amyloid beta, where a higher quotient of total minus monomeric amyloid beta over total amyloid beta provides an indication of greater neurodegeneration risk, greater likelihood of presence of degenerative disease, or deteriorating condition of the subject.

Some methods determine an amount of oligomeric amyloid beta, where a higher amount of oligomeric amyloid beta provides an indication of greater neurodegeneration risk, greater likelihood of presence of degenerative disease, or deteriorating condition of the subject.

Methods of the invention involve obtaining a sample from a subject to be tested. The sample may include tissue or body fluid that is suspected to include an analyte of interest, such as amyloid beta monomers or oligomers. In general, a body fluid is a liquid material derived from a patient or non-human subject. Such body fluids include, but are not limited to, blood, plasma, serum, and serum derivatives. In preferred embodiments, the body fluid sample is a cerebrospinal fluid sample, such as lumbar or ventricular cerebrospinal fluid.

Amyloid beta can adsorb to certain materials. Therefore, excepting the needle, only polypropylene tubing and collection kit components are acceptable. The sample may be collected in any clinically acceptable manner. A sample may be a fine needle aspirate or liquid biopsied sample, e.g., a spinal fluid aspirate, or the like. A sample also may be media containing cells or biological material. A sample may also be plasma obtained from whole blood in an EDTA collection tube. To ensure sample stability, centrifuge plasma samples, then both CSF and plasma should be aliquoted, labeled and frozen within 60 minutes of collection and stored at approximately −70° C. until tested.

In some embodiments, measuring an amount of amyloid beta carboxyl terminus available for binding and measuring an amount of amyloid beta amino terminus in the sample is performed simultaneously on a single sample. In some embodiments, measuring an amount of one or more amyloid beta carboxyl terminus available for binding representing monomer and measuring an amount of amyloid beta amino terminus in the sample is performed simultaneously on a single sample. In other embodiments, the measurements are taken from different aliquots from a single sample. In other embodiments, the measurements are taken from separate samples.

Methods of the invention are useful for detecting and characterizing amyloid beta in a subject. In some cases the subject is a mammal, such as a human. A subject can be any human suspected of having or believed to have a likelihood of developing Alzheimer's disease. In some embodiments the subject may be a candidate for entry into a clinical trial, such as to test a drug for treatment or prophylaxis of Alzheimer's disease. Methods of the invention as described herein can be used to screen for clinical trial participation criteria. For example, if the quotient of monomeric amyloid beta over monomeric and oligomeric amyloid beta is below a threshold, the subject is included in the clinical trial, and if the subject is above the threshold the subject is excluded from the clinical trial. Some methods further comprise informing the subject or a care provider of the subject of the diagnosis, prognosis, or monitoring. Methods of the invention as described herein can be used as a pre-clinical screen of potential therapeutics to identify those with significant effects relative to placebo control in non-human subjects e.g. transgenic rodents.

In some embodiments, methods of the invention can be used in conjunction with population screening, and the subject can thus be one from a population to be screened for Mild Cognitive Impairment or Alzheimer's disease. The methods can be used to determine which subjects in a population to administer a drug to effect prophylaxis or treatment for Mild Cognitive Impairment or Alzheimer's disease. Such methods comprise for each subject in the population: (a) measuring an amount of monomeric amyloid in a sample of body fluid; (b) measuring an amount of monomeric and oligomeric amyloid beta in a second sample of the body fluid; and (c) comparing the amounts of monomeric amyloid beta to monomeric and oligomeric amyloid beta. Optionally, the method further includes administering a drug or treatment to a subset of the population based on the comparison, wherein the drug or treatment is to effect prophylaxis for Alzheimer's disease. In some methods, the comparison determines a ratio between monomeric amyloid beta and total amyloid beta (i.e., monomeric plus oligomeric amyloid beta), and subjects in which the quotient of the monomeric amyloid beta over total amyloid beta ratio is below a threshold, receive the drug.

The invention further provides methods of determining which subjects in a population to enroll in a clinical trial, comprising for each subject in the population: (a) measuring an amount of monomeric amyloid beta in a sample of body fluid; (b) measuring an amount of monomeric and oligomeric amyloid beta in a second sample of the body fluid; and (c) comparing the amount of monomeric amyloid beta to monomeric and oligomeric amyloid beta, wherein subject(s) in the population are or are not enrolled in the clinical trial based on the comparison. In some methods, the comparing determines a ratio between monomeric amyloid beta and monomeric and oligomeric amyloid beta and subjects in which the quotient of monomeric amyloid beta over monomeric and oligomeric amyloid beta falls below a threshold are enrolled in the clinical trial.

Subjects can be assessed independent of their present cognitive status using methods of the invention. In some methods, the subject does not have cognitive impairment and the step of quantifying soluble diffusible neurotoxic amyloid beta in the subject is for the purpose of assessing the subject's susceptibility to developing Alzheimer's disease. In some methods, the subject has mild cognitive impairment and the step of quantifying soluble diffusible neurotoxic amyloid beta in the subject is for the purpose of assessing the subject's susceptibility to developing Alzheimer's disease.

The comparison of monomeric amyloid beta and total amyloid beta may be used in conjunction with other data points for determining a subject's risk of developing AD. For example, if the subject has mild cognitive impairment, using the comparison between monomeric and total amyloid to quantify soluble diffusible neurotoxic amyloid beta in the subject sample can be combined with other symptoms and signs of the subject's condition to provide a diagnosis of Alzheimer's disease. In some embodiments, the subject has been diagnosed with Alzheimer's disease before performing the method and the quantification provides an indication of the stage of the disease. In some embodiments, the subject is receiving treatment or prophylaxis for Alzheimer's disease, and the quantification provides an indication of the subject's response to treatment. In some embodiments, the method is performed at intervals and a change in the comparison between monomeric and total amyloid over time provides an indication of the response to treatment.

In other embodiments, the methods described herein can be used on non-human subjects, such as in preclinical screening. The invention is useful for screening an agent for activity against Alzheimer's disease in a model organism. A method for screening an agent for activity against Alzheimer's disease may comprise: (a) contacting a transgenic rodent model of Alzheimer's disease with the agent; (b) comparing the amount of monomeric amyloid beta to the amount of total amyloid beta (i.e., monomeric and oligomeric amyloid beta) in a body fluid of the transgenic rodent contacted with the agent; and (c) using the comparison in determining whether the agent has activity useful in treating Alzheimer's disease.

Methods of the invention involve antibody-based detection and analysis. In order to quantify amyloid oligomers, particular epitopes of amyloid beta identification of microorganisms includes the use of antibody-based detection methods. Antibody-based detection methods are generally based on the transformation of a specific biomolecular interaction between antigen and antibody into a macroscopically detectable signal or change in the physical properties of the media. See e.g., Sveshnikov, Peter; “The Potential of Different Biotechnology Methods in BTW Agent Detection: Antibody Based Methods” The Role of Biotechnology in Countering BTW Agents; Vol. 34 of the series NATO Science Series, pp. 69-77 (2001), incorporated herein by reference.

Particular antibody detection methods are known in the art. Proteins can be detected and quantified through epitopes recognized by polyclonal and/or monoclonal antibodies used in methods such as enzyme-linked immunoabsorbent assay (ELISA), immunoblot assays, flow cytometric assays, radioimmuno assays, immunocytochemical assays, Western blot assays, an immunofluorescent assays, chemiluminescent assays, flow cytometry and fluorescence-activated cell sorting (FACS), immunoprecipitation, enzyme linked immunospot (ELISPOT), and other polypeptide detection strategies. Proteins may also be detected by mass spectrometry assays (potentially coupled to immunoaffinity assays) including matrix-assisted laser desorption/ionization time-of-flight (MALDI-TOF) mass mapping and liquid chromatography/quadrupole time-of-flight electro spray ionization tandem mass spectrometry (LC/Q-TOF-ESI-MS/MS). Additionally, methods of the disclosed invention may include tagging of proteins separated by two-dimensional polyacrylamide gel electrophoresis (2D-PAGE), (Kiernan et al, Anal Biochem 301, 49-56 (2002); Poutanen et al, Mass Spectrom 15, 1685-1692 (2001) the content of each of which is incorporated by reference herein in its entirety) or any other method of detecting protein.

In some embodiments, determination of epitope binding levels can be determined by constructing an antibody microarray in which binding sites comprise immobilized, preferably monoclonal, antibodies specific to a plurality of protein species encoded by the cell genome. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In some embodiments, immunohistochemistry methods may be used for detecting the presence of certain epitopes. In these methods, antibodies (monoclonal or polyclonal) specific for each marker are used to detect expression. Immunohistochemistry protocols and kits are well known in the art and are commercially available.

The present invention contemplates using antibody-based detection methods to quantify both carboxyl and amino termini of amyloid beta. The invention relies first on the ability to measure the amount of monomeric amyloid beta using antibodies that bind to carboxyl terminal epitopes that are sterically available in monomeric amyloid beta but which are not sterically available in oligomeric amyloid beta. Various carboxyl amyloid beta methods are contemplated by the present invention. In some embodiments, at least one of Aβx-37, Aβx-38, Aβx-39, Aβx-40, Aβx-41, and Aβx-42 are measured. Some methods measure at least Aβx-40. Some methods measure at least Aβx-42. Some methods measure at least Aβx-40 and Aβx-42.

In some methods, the amount of monomeric amyloid beta is measured using one or more antibodies that bind to one or more carboxyl terminal epitopes present in monomeric amyloid beta and not present in oligomeric amyloid beta. In some methods, the one or more carboxyl terminal antibodies comprise one or more end-specific antibodies for Aβ37, Aβ38, Aβ39, Aβ40, Aβ41, or Aβ42. In some methods, one or more carboxyl terminal antibodies includes an antibody that is end-specific for Aβ40, optionally antibody (e.g. commercial reagents 2G3, MABN11). In some methods, the one or more carboxyl terminal antibodies includes an antibody that is end-specific for Aβ42, optionally commercial antibody MABN12, MABN13 or 05-831-1. In some methods, the one or more carboxyl terminal antibodies includes both an antibody that is end specific for Aβ40 and an antibody that is end-specific for Aβ42.

In some methods the monomeric amyloid beta is measured by an immunoaffinity sandwich assay including the one or more carboxyl terminal antibodies and another antibody that binds to an amino terminal and/or central epitope. In some methods, the other antibody binds to an amino terminal epitope, optionally wherein a commercial antibody: e.g. AHP1252, MABN639, and 1E8 from Biorad, Millipore and Sigma, respectively. In some methods, the other antibody binds to a central epitope optionally wherein the antibody is e.g. commercial reagents 6E10 or 4G8 or proprietary antibody 266. In some methods, the one or more carboxyl terminal antibodies are reporter antibodies and the other antibody is a capture antibody. In some methods, the one or more carboxyl terminal antibodies are capture antibodies and the other antibody is a reporter antibody. In some ECL methods, the one or more reporter antibodies are labeled with ruthenium and the capture antibody is labeled with biotin or an affinity tag. In some ELISA methods, the one or more reporter antibodies are labeled with and enzyme and the capture antibody is labeled with biotin or an affinity tag, or is adsorbed and immobilized onto a surface.

In some methods to determine the amino terminal concentration, the amount of total amyloid beta is measured using one or more antibodies that bind to one or more amino terminal epitopes present in total amyloid beta. In some methods, the one or more amino terminal antibodies are one or more end-specific antibodies for Aβ1, Aβ2, Aβ3, Aβ4, Aβ5, Aβ6, Aβ7, Aβ8, Aβ9, Aβ10, or Aβ11. In some methods, one or more amino terminal antibodies including antibody end-specific for Aβ1-5, optionally 3D6 or bapineuzumab. In some methods the total amyloid beta is measured by an immunoaffinity sandwich assay including the one or more amino terminal antibodies and another antibody that binds to a carboxyl terminal and/or central epitope, optionally 6E10 or 4G8. In some methods, the other antibody binds to an amino terminal epitope, optionally wherein the antibody is 3D6. In some methods, the other antibody binds to a central epitope optionally wherein the antibody is 266. In some methods, the one or more amino terminal antibodies are reporter antibodies and the other antibody is a capture antibody. In some methods, the one or more amino terminal antibodies are capture antibodies and the other antibody is a reporter antibody. In some ECL methods, the one or more reporter antibodies are labeled with ruthenium and the capture antibody is labeled with biotin or an affinity tag. In some ELISA methods, the one or more reporter antibodies are labeled with an enzyme and the capture antibody is labeled with biotin or an affinity tag.

Appropriate method controls are required in order to enable comparison of separate amyloid beta and monomer amyloid beta concentrations in a GLP or GMP testing laboratory. Qualification of new reagent lots and demonstrating stability of critical method reagents is required. The preferred determination of available amyloid beta termini measures the amino (Site N) and carboxyl (Site C) concentration against the same set of amyloid reference standards used to generate the standard response curve. The preferred method will also include a range of Quality Controls (Reference Standard spiked into negative CSF matrix), and utilize the same manufacturer's lot of reporter molecule which binds centrally (Site B) to the amyloid beta peptide. The relationship between an amino based standard curve and the carboxyl based standard curves and the estimates of quality control samples (reference standards spiked into background sample matrix prepared at three levels across the range of the standard curve concentrations) can be established by multiple runs of standard curves using the same reference standard. While the specific affinity/avidity of each immunoreagent may effect results, typically, reagent monoclonal antibodies are high affinity, method incubation times allow complete binding, yield stable immunocomplexes, that will generate comparable dose response curves, and therein comparability of results. To demonstrate comparability of reference standard curve response between total and monomer, monomer specific antibody reagents (site N and site B) with reference standard and total specific antibody reagents with reference standard can be determined in one run (or independent runs) each on half of an 8×12 well plate. For example, in an electrochemiluminescence (ECL) protocol, coincubate biotinylated capture antibody to Site C and reporter Ru-antibody to site B with the set of Reference Standard Curve concentrations. In parallel, coincubate biotinylated capture antibody to the Site N and preferred, the same reporter Ru-antibody to site B with the set of Reference Standard Curve concentrations. Commercial Streptavidin coated ECL plates capture biotinylated antibody-Reference Standard-reporter-ruthenium complexes. Signal generated from the amino and carboxyl amyloid determinations are proportional to peptide concentration are assessed for comparability or to establish a mathematical relationship between determination methods. Alternatively, standards and samples for monomer and total amyloid beta are run on separate plates. Alternatively, total and monomer determinations can be performed by different techniques with reduced comparability and reliability of data interpretation.

### Immunotherapy Interference

Methods of the invention can be used to measure samples from a subject undergoing immunotherapy treatment against amyloid beta. To do so, the immunotherapeutic interacting with amyloid beta must first be neutralized in the sample before measuring the amyloid beta termini. For example, in some embodiments the subject is being treated with a monoclonal antibody immunotherapy against amyloid beta amino terminus, such as bapineuzumab or a mid binding immunotherapeutic such as solanuzumab. In this case, the sample is treated with an anti-idiotype antibody before being assayed for amyloid beta e.g. JH11.22G2 is an anti-idiotype for bapineuzumab. The appropriate anti-iodiotype will reduce or eliminate the interfering effect of the therapeutic molecule on the measurement of amyloid beta.

Sample matrix interference of amyloid beta spike and recovery in plasma samples improves with slightly denaturing conditions. Plasma samples are mixed with guanidine buffer to improve recovery of amyloid beta reference standard spiked into negative control plasma (matrix). Some methods further comprise determining an amount of Tau or P-Tau relative to a control value, which provides a further indication of susceptibility to developing Alzheimer's disease, presence of Alzheimer's disease, or deteriorating condition of the subject.

### Diagnostic Kits

In certain embodiments the invention involves a diagnostic kit configured to perform any of the methods described herein. A diagnostic kit may include at least a carboxyl terminal antibody, an amino terminal antibody, and a center-specific antibody. The carboxyl terminal antibody is end-specific for Aβ37, Aβ38, Aβ39, Aβ40, Aβ41, or Aβ42. The amino terminal antibody is end-specific for Aβ1, Aβ2, Aβ3, Aβ4, Aβ5, Aβ6, Aβ7, Aβ8, Aβ9 or Aβ10. The center-specific antibody is specific for a central epitope of AP. In some kits, the carboxyl terminal antibody is end-specific for Aβ40 or Aβ42. Some kits include a carboxyl terminal antibody end-specific for Aβ40 and a carboxyl terminal antibody end-specific for Aβ42. The kit containing these antibodies would be able to perform the various methods described herein, as would be understood by a person of ordinary skill in the art.

### Difference Calculation

Amyloid beta oligomer measurements from an initial sample (Time 1) can be compared to those in a second sample (Time 2). This can be done, for example, to monitor a patient's susceptibility to Alzheimer's disease over time, track the progression of the disease, or monitor the efficacy of a treatment regimen. Ideally test both samples in the same run. In a particular embodiment of the method: for both samples, (a) an amount of monomeric amyloid beta in a sample of body fluid is measured; and (b) an amount of monomeric and oligomeric amyloid beta in a sample of the body fluid is measured. For each sample the difference of (b) minus (a) represents oligomer amyloid beta. Results between Time 1 and Time 2 are compared. Increasing amyloid beta oligomer indicates an increasing susceptibility to Alzheimer's disease and/or neurological risk. Some methods further comprise using the calculated ratio or difference for providing a diagnosis, a prognosis, or monitoring of Alzheimer's disease or susceptibility thereto in the subject. A lower quotient of the amount in step (a) and step (b) or a higher difference between the amount in step (b) and step (a) indicates greater susceptibility to developing the disease, greater likelihood of presence of the disease, or deteriorating condition of the subject.

### Systems

Antibody-based detection, diagnosis, and monitoring methods described herein can be performed on systems that incorporate a computing device, such as a computer, that includes a processor, e.g., a central processing unit, or any combination of computing devices where each device performs at least part of the process or method. Methods of the invention can be performed using software, hardware, firmware, hardwiring, or combinations of any of these.

Instruments for obtaining or analyzing antibody-based detection data may include or be connected to processors. Processors suitable for the execution of computer programs for use with the invention include, by way of example, both general and special purpose microprocessors, and any one or more processor of any kind of digital computer. Generally, a processor will receive instructions and data from a read-only memory or a random access memory or both. The essential elements of a computer for use with the invention are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. The processor and the memory can be supplemented by, or incorporated in, special purpose logic circuitry.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, a reference set of data may be stored at a remote location, such as in a reference database, and the computer communicates across a network to access the reference set to compare data derived from an individual patient to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The methods of antibody-based analysis described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers).

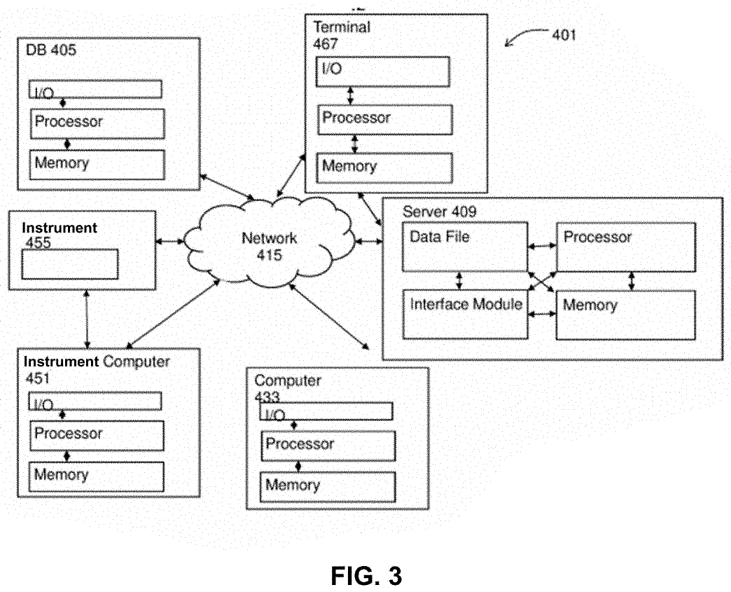

In an exemplary embodiment shown in FIG. 3, system 401 can include a computer 433 (e.g., laptop, desktop, or tablet). The computer 433 may be configured to communicate across a network 415. Computer 433 includes one or more processor and memory as well as an input/output mechanism. Where methods of the invention employ a client/server architecture, any steps of methods of the invention may be performed using server 409, which includes one or more of processor and memory, capable of obtaining data, instructions, etc., or providing results via interface module or providing results as a file. Server 409 may be engaged over network 415 through computer 433 or terminal 467, or server 415 may be directly connected to terminal 467, including one or more processor and memory, as well as input/output mechanism. In some embodiments, systems include an instrument 455 for obtaining antibody-based detection data, which may be coupled to a computer 451 for initial processing.

Memory according to the invention can include a machine-readable medium on which is stored one or more sets of instructions (e.g., software) embodying any one or more of the methodologies or functions described herein for determining amyloid concentrations and/or making a diagnosis and/or identifying a neurodegenerative risk in a patient. The software may also reside, completely or at least partially, within the main memory and/or within the processor during execution thereof by the computer system, the main memory and the processor also constituting machine-readable media. The software may further be transmitted or received over a network via the network interface device.

### INCORPORATION BY REFERENCE

Any and all references and citations to other documents, such as patents, patent applications, patent publications, journals, books, papers, and web contents, which have been made throughout this disclosure, are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

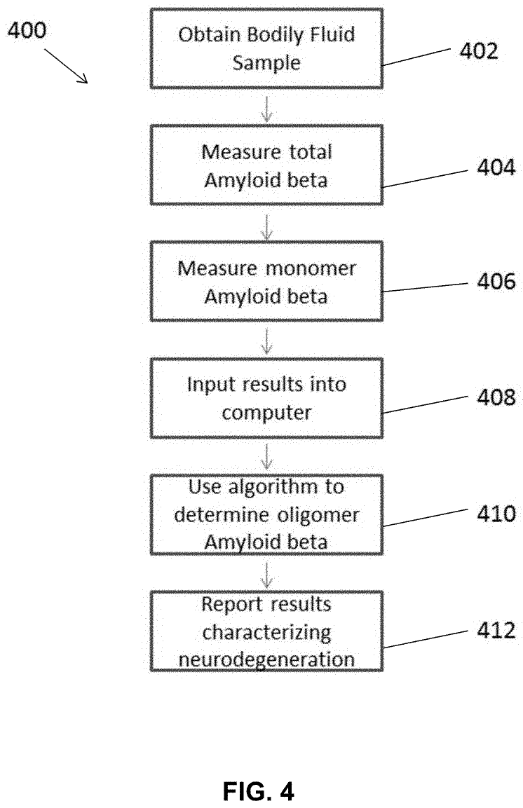

The following is a general approach for a quantification assay according to the present disclosure. A method known as AMRA (Amyloid beta termini Ratio) is designed to quantify amyloid beta (Aβ) peptide concentrations in human cerebrospinal fluid (CSF) using immunoreagents targeting amyloid beta terminal epitopes. Monomeric amyloid beta, but not oligomeric amyloid beta, is available to immunoreagent binding at the carboxyl terminus. Therefore, the observed amyloid beta x-42 represents the amount of monomeric amyloid beta. However, both monomeric amyloid beta and soluble oligomeric amyloid beta are available to immunoreagent binding at both amino and mid binding antigen loci. Therefore, the observed amyloid beta 1-x represents the total amount of amyloid beta. The Amyloid Ratio (AMRA), which is amyloid beta x-42 to amyloid beta 1-x represents the ratio of monomer amyloid to total amyloid. When the ratio is approximately 1, the interpretation is that all amyloid in the sample is monomeric and there is no present risk of neurodegeneration. However, if the AMRA result is substantially below 1, such a result indicates/suggests that a percentage of amyloid in the sample is soluble amyloid beta oligomers, demonstrated neurotoxins that cause neurodegeneration. The process flow is shown generally in FIG. 4. The method 400 of quantifying the extent of oligomer aggregation of amyloid beta begins with a step 402 of obtaining a bodily fluid sample from a subject. The bodily fluid sample may be blood, plasma, or cerebral spinal fluid sample. In step 404, the total Amyloid beta is measured. In one embodiment the measurement includes determining the concentration of amyloid beta amino termini. In step 406, the monomer Amyloid beta is measured. In one embodiment the measurement includes determining the concentration of amyloid beta carboxyl termini. Step 404 and step 406 may be interchanged, as the order is not pertinent to the results obtained. The results of steps 404 and 406 are input into a computing device in step 408. In step 410 an algorithm is run that generates a number that quantifies the extent of oligomer amyloid beta. The algorithm may be a difference of the results of steps 404 and 406, a ratio of the results of steps 404 and 406, or a ratio of the sum or differences of steps 404 and 406. These are example algorithms for illustrative purposes and are not meant to be exhaustive. It is clear to one skilled in the art that other possible algorithms may be applied to generate a unique number. The result of the algorithm of step 410 is used in step 412 to characterize amyloid beta oligomer representing a risk of neurodegeneration in the subject from whom the sample was obtained in step 402. For example, if the algorithm of step 410 is a ratio of the results of step 406 over the results of step 404, then a result in step 412 of a value of 1 indicates that there is not an appreciable amount of oligomer amyloid beta, and the subject does not have appreciable neurodegeneration. In other words, this result indicates a normal subject. If the ratio is significantly greater than 1, that indicates the presence of oligomer amyloid beta, and the presence of neurodegeneration risk in the subject.

### Example 1

This fluorometric immunoassay determines the amino terminal specific amyloid beta 1-x concentration, representing total amyloid beta and determines the carboxyl terminal specific amyloid beta x-42 concentration, representing monomeric amyloid beta in cerebrospinal fluid. These results combined characterize the molecular population of amyloid beta, including quantification of soluble amyloid beta oligomers. Fluorometric based methods may use but are not limited to Luminex 100 IS Total System, Luminex 200 IS Total System or BioPlex 200 System for the simultaneous quantification of multiple analytes, e.g. either of amyloid beta (1-x or x-42) determination with TAU and/or P-TAU determinations). Carboxyl and amino amyloid beta concentrations are determined from replicate samples. Different analytes are selectively immobilized onto beads of a specific region number by a capture monoclonal antibody (AT270 for pTAU, AT120 for TAU, and 4D7A3 for Amyloid beta x-42, or 3D6 for Amyloid beta 1-x) coupled covalently to microspheres. A mix of microspheres with capture antibodies is incubated with CSF samples or standards in a filter plate, washed collected then suspended together with a mix of biotinylated reporter antibodies. Each biotinylated antibody detects one or several parameters (e.g. B-266 for amyloid beta 1-42, B-HT7 for detection of pTAU and TAU). The antigen-antibody complex is detected by a phycoerythrin-labeled streptavidin complex. After a wash step the solution is immediately measured in a Luminex 100 IS Total System. The fluorescence intensity on a specific bead is related to the concentration of the parameter for which it was designed. Based on its fluorescent signature each microsphere is accurately classified to its own unique region number. In addition, each bead is scanned individually for the presence of a reporter fluorescence that quantifies the analyte at the surface of the bead. Determination of amyloid beta amino (total) and carboxyl termini (monomer) characterize the proportion of amyloid beta molecular populations in the sample, deducing amyloid beta oligomer concentration from a difference: total amyloid minus monomer amyloid (e.g. amyloid beta amino minus amyloid beta carboxyl concentration). A higher amyloid beta oligomer value represents greater risk of current neurodegeneration. Alternatively the ratio of amyloid monomer to total amyloid (carboxyl/amino) represents the proportion of amyloid beta present as monomer. A higher value of monomer represents lower risk, a normal individual. The ratio may be indicative of a particular risk level for the patient or a level of progression of disease. For example, a ratio of 0.90 or above may be considered low risk and/or no significant neurodegeneration. A ratio of 0.70-0.90 may be considered moderate risk and/or moderate neurodegeneration; and a ratio below 0.70 may be considered high risk and/or advanced neurodegeneration. Those of skill in the art may define more or fewer categories of risk, and the categories may have different boundaries than those listed in the example above. In all cases, however, as the ratio recedes further from 1, it represents a greater proportion of soluble aggregate amyloid. Alternatively, the ratio of a difference (total-monomer/total) calculates proportion of amyloid beta oligomer versus total amyloid beta, a lower value representing increasing risk of oligomer mediated neurodegeneration. The range of ratio values depends on monomer measures. Amyloid beta 1-42 and 1-40 are the predominant peptides. Measure 1-42 excludes 1-40 peptides measured by the total amyloid beta. The combination of additional biomarkers such as TAU or pTAU, brain imaging techniques, and/or psychometric tests may provide means for the early detection, diagnosis, monitoring or assessing potential therapeutic effects.

### Example 2

This procedure is an Enzyme Linked Immunosorbent Assay (ELISA) to quantify amyloid beta in cerebrospinal fluid by amino terminal specific and carboxyl terminal specific methods and then to quantify soluble amyloid beta oligomer concentration. This colorimetric ELISA uses unique capture antibodies (3D6 and 21F12 specific for amino and carboxyl termini respectively) and a common reporter antibody: Horse radish peroxidase conjugated to mid amyloid beta specific antibody 266 (HRP-266). Ideally, methods use the same lot of reporter reagent. Determinations of amino amyloid beta use 3D6 and HRP-266), and the carboxyl amyloid beta determinations use 21F12 and HRP-266. Each step is a 50 minute room temperature rotating incubation then washing to remove non-specifically bound ligands. Capture antibodies are bound onto wells of microtiter plates (e.g. Costar Cat. No. 3590), other well binding sites are blocked with non-specific protein. Not all capture antibodies bind equally to microtiter plates potentially altering ligand binding capacity. Alternatively, biotinylated capture antibodies bound onto streptavidin or avidin microtiter plates may improve binding and functional equivalence. Add and incubate samples, standards and controls. Wash the plate add enzyme antibody HRP-266. Add tetramethylbenzidine (TMB) as enzyme substrate and chromophore. The reaction is stopped by adding TMB stop solution, and the optical density (OD) values are measured spectrophotometrically. SoftMax Pro software is used to control the spectrophotometer, for data reduction and analysis. As described previously, algorithms applied to the values for amino (representing Total amyloid beta) and carboxyl (representing Monomer amyloid beta) determinations in a ratio of monomer/total reveal the percentage or proportion of amyloid beta monomers, a greater value reflecting low risk of neurodegeneration. Importantly, a lower value representing greater amount of amyloid beta oligomer representing an increased risk of amyloid beta mediated neurodegeneration. Controls include Negative CSF Matrix, Amyloid beta Reference Standard spiked into Negative Control CSF at three concentrations across the range of the Standard Curve. The Standard Curve comprises amyloid beta Reference Standard added at 6-8 concentrations to a defined buffer. Remove/reduce endogenous amyloid beta from CSF by brief incubation with polystyrene.

### Example 3

This procedure is an electrochemilumenescence (ECL) immunoassay to characterize amyloid beta in cerebrospinal fluid by quantifying amino terminal specific (amyloid beta 1-x) and quantifying carboxyl terminal specific (amyloid beta x-40) methods. Those results determine the soluble amyloid beta oligomer concentration.

The amyloid beta 1-x assay measures the concentration of amyloid beta 1-x in human CSF. Two labeled anti-amyloid beta mouse monoclonal antibodies are co-incubated with the standards, controls and test samples at least neat and one further dilution in duplicate, to form immunocomplexes. During this incubation the 96 well plate only serves as a container. One of the labeled antibodies is a biotinylated mouse monoclonal anti-amyloid beta antibody from clone 3D6 that is specific to the N-terminus of the amyloid beta 1-x. The second reporter antibody is a ruthenium linked mouse antibody specific to the mid amyloid beta region Site B e.g. Ru-266 (FIG. 2). After incubation, the standards, controls and test samples are transferred to a MSD 96 well streptavidin coated microplate and incubated for one hour at room temperature to capture immunocomplexes. The plate is then washed and MSD read buffer containing ECL catalyst triproplyamine is added. The plate is read on a MSD Sector 2400 instrument which applies voltage to electrodes on the bottom of the MSD plate and generates ECL signal. The signal is directly proportional to the concentration of amyloid beta 1-x present in calibration standards and samples. Results for unknowns are reported as the mean concentration of duplicate wells. Data analysis is performed using SoftMax Pro using a 4-parameter logistic fit regression equation.

The amyloid beta x-40 assay measures the concentration of amyloid beta x-40 in human CSF. Two labeled anti-amyloid beta mouse monoclonal antibodies are co-incubated with the standards, controls and test samples at least neat and one further dilution in duplicate, to form immunocomplexes. During this incubation the 96 well plate only serves as a container. One of the labeled antibodies is a biotinylated mouse monoclonal anti-amyloid beta antibody from clone 2G3 that is specific to the C-terminus of the amyloid beta x-40. The second reporter antibody is a ruthenium linked mouse antibody specific to the mid amyloid beta Site B (e.g. Ru-266) (FIG. 2). Ideally, the same lot of reporter reagent is used in both determinations. After incubation, the standards, controls and test samples are transferred to a MSD 96 well streptavidin coated microplate and incubated for one hour at room temperature to capture immunocomplexes. The plate is then washed and MSD read buffer containing ECL catalyst triproplyamine is added. The plate is read on a MSD Sector 2400 instrument which applies voltage to electrodes on the bottom of the MSD plate and generates ECL signal. The signal is directly proportional to the concentration of amyloid beta x-40 present in calibration standards and samples. Data analysis is performed using SoftMax Pro using a 4-parameter logistic fit regression equation. Unknown CSF sample concentration is back calculated from signal/response of the Standard Curve only if run acceptance criteria are met. The Standard Curve and Quality Control precision and accuracy serve as run acceptance criteria. Results for unknowns are reported as the mean concentration of duplicate wells.

A kit of critical reagents and instructions could be used to screen subjects for presence or amount or proportion of soluble amyloid beta oligomers in body fluid. A kit with instructions and reagents specific for fluorometric, colorimetric, enzymatic, electrochemilumenesence, or radioisotopic immunoassay is proposed for population screening, those with genetic risk factors, cognitively normal individuals, setting risk ratio values among control or normal individual and those with some clinical progression including Mild Cognitive Impairment and Alzheimer's Disease.

### Enhanced Example 3B

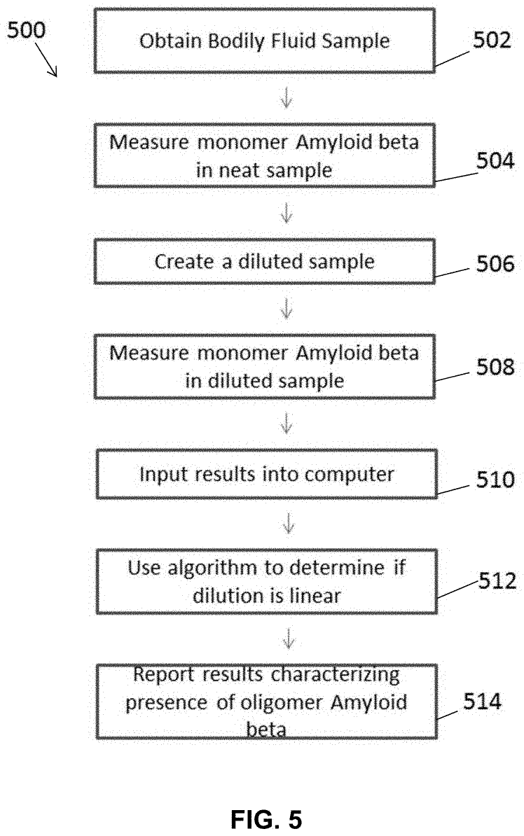

An enhanced AMRA method may be conducted when amyloid samples are tested neat and at a second dilution, as shown in FIG. 5. Bioanalytical methods require validation per Code of Federal Regulation (CFR) for use in Good Lab Practice (GLP) or for Quality Control in current Good Manufacturing Practice (cGMP). Validation parameters include Accuracy, Precision, Specificity, Selectivity, Limit of Detection, Limit of Quantitation, Range, and Stability. Matrix interference defines the initial sample matrix dilution required or needed to dilute an interfering substance. When dilutions are tested, validation acceptance criteria of Dilutional Linearity assess if all dilutions closely estimate the back calculated neat analyte concentration. Amyloid assays often fail Dilutional Linearity. Often, more dilute samples generate a higher analyte concentration estimates. Thus the assay fails precision acceptance criteria. As a result, amyloid assays use methods with no dilution and/or methods with greatest dynamic range. Amyloid equilibria explain this result. Typically in subjects with Mild Cognitive Impairment or Alzheimer's Disease, CSF at neat concentration contains both amyloid monomers and soluble amyloid multimers (ADDL). Current amyloid beta assays measure monomer (amyloid beta x-42 or x-40). Dilution of CSF will lower the amyloid monomer concentration resulting in re-equilibrium among the population of amyloid beta molecules. Some soluble amyloid oligomers will dissociate into monomers, thus increasing the measured amyloid and concomitant higher neat amyloid estimate. The non-linearity of dilutions is taken into consideration in this example. Dilutional non-linearity monomer estimates are used to further demonstrate the presence of oligomers in dynamic equilibrium that dissociate upon dilution from multimers into monomers. Amino quantitation and carboxyl quantitation assays can be evaluated in a step-wise manner as described below.

An amino quantitation where total neat vs total dilute meet the Dilutional Linearity acceptance criteria is an expected result, as the amino-based measure represents the hydrophilic terminus. The carboxyl quantitation may or may not meet the dilutional linearity acceptance criteria. When the carboxyl quantitation reveals Monomer neat vs Monomer dilute meets Dilutional Linearity acceptance criteria, these data suggest the sample contains mostly monomer amyloid beta. As a result, the patient would be considered low risk for Alzheimer's disease or neurodegeneration. When the carboxyl quantitation shows the monomer neat vs monomer dilute does not meet the Dilutional Linearity acceptance criteria, these data suggest a demonstrable level of amyloid beta oligomer. Diluted amyloid beta oligomers will dissociate to the new equilibrium increasing the quantified total amyloid beta. This may be interpreted as a neurodegeneration risk due to presence of soluble amyloid beta oligomers. This result could be criteria for inclusion/exclusion in a preventative, prophylactic clinical trial, or screening outcome for risk assessment in a population of subjects.

A combination cross-assay result may be indicative of a particular risk to the subject. For example, an amino quantitation where total neat vs total dilute meet the Dilutional Linearity acceptance criteria; combined with a carboxyl quantitation where Monomer neat vs Monomer dilute meet Dilutional Linearity acceptance criteria, these data suggest the sample contains mostly monomeric amyloid beta. This expected result indicates low risk of Alzheimer's disease, or in other words, a normal subject.

In another example, if the results show an amino quantitation where total neat vs total dilute meet Dilutional Linearity acceptance criteria (the expected result) and a carboxyl quantitation where monomer neat vs monomer dilute does not meet Dilutional Linearity acceptance criteria, then these data suggest a non-zero level of amyloid beta oligomer. The resulting comparison between monomeric amyloid beta and total amyloid beta provide an estimate of soluble amyloid beta oligomer concentration. The relative neurodegeneration risk increases as the monomer/total amyloid beta ratio falls. The amyloid equilibria concept has more evidence when more dilute carboxyl (monomer) samples yield higher amyloid beta monomer estimates. In cases where dilute CSF and neat CSF have close estimates, it is likely that no amyloid multimer (toxic amyloid) is present. Non-dilutional linearity of amyloid beta monomer alone demonstrates presence of soluble oligomeric amyloid beta, and indicates some risk of amyloid beta mediated neurotoxicity.

FIG. 5 diagrams the enhanced AMRA method 500 for determining the presence of neurodegeneration in a subject. In step 502, a bodily fluid sample is obtained from a subject. The bodily fluid sample may be blood, plasma, or cerebral spinal fluid sample. In step 504, the monomer Amyloid beta is measured. In one embodiment the measurement includes determining the concentration of amyloid beta carboxyl termini. Other methods may be used to obtain this measurement, and the examples used here are for illustrative purposes and are not limiting to the scope of the patent. Step 506 includes a process to create a diluted sample of the bodily sample fluid obtained in step 502. Step 508 includes further measuring the monomer Amyloid beta in the diluted sample of step 506. The method of measuring the Amyloid beta monomer may be the same or different method as used in step 504. The results of the measurements of step 504 and step 508 are input into a computing device, and an algorithm is used to determine if the sample follows dilutional linearity accuracy and precision acceptance criteria; i.e., back-calculated concentration of neat and diluted replicate (precision) samples must closely estimate the same value (accuracy). If the diluted sample neat estimate is higher than the neat determination the sample exhibits nonlinear dilution characteristics. The sample obtained from the subject contains oligomeric amyloid beta. The extent of nonlinearity may be used to characterize the extent of neurodegeneration. It is clear to those skilled in the art the steps 506 and 508 may be repeated at multiple dilutions to further quantify the extent of neurodegeneration.

### Example 4

This method first measures amyloid beta x-40 concentration and second measures amyloid beta 1-x in human plasma.

In a first test, amyloid beta carboxyl terminal (amyloid beta x-40), concentration is determined by electrochemilumenescence (ECL), using the Meso Scale Discovery (MSD) Sector Imager 2400 and commercial reagents for conjugation and assay runs. To improve recovery, plasma samples are initially incubated in a guanidine containing buffer. After sample treatment, two labeled anti-amyloid beta monoclonal antibodies are co-incubated for 3 hours at ambient temperature with the standards, controls and test samples to form immunocomplexes. One of the labeled antibodies is a biotinylated mouse monoclonal to site C. The second antibody is a ruthenium conjugate specific to site B (FIG. 2). The mixtures are transferred to a MSD streptavidin plate which capture immunocomplexes via biotin ligands. The plate is washed and Read Buffer containing ECL catalyst tripropylamine is added. Using the MSD Sector Imager, an applied voltage generates an ECL signal. The signal is proportional to the concentration of amyloid beta x-40 present in the plasma. Results for unknowns are reported as the mean concentration of duplicate wells. Data analysis is performed with SoftMax Pro using a 4-parameter logistic fit regression equation.

Secondly, amyloid beta amino terminal (amyloid beta 1-x), concentration is determined by electrochemilumenescence (ECL), using the Meso Scale Discovery (MSD) Sector Imager 2400 and commercial reagents for conjugation and assay runs. To improve recovery, plasma samples are initially incubated in a guanidine containing buffer. After sample treatment, two labeled anti-amyloid beta monoclonal antibodies are co-incubated for 3 hours at ambient temperature with the standards, controls and test samples to form immunocomplexes. One of the labeled antibodies is a biotinylated mouse monoclonal to site N. The second antibody is a ruthenium conjugate specific to site B (FIG. 2). The mixtures are transferred to a MSD streptavidin plate which capture immunocomplexes via biotin ligands. The plate is washed and Read Buffer containing ECL catalyst tripropylamine is added. Using the MSD Sector Imager, an applied voltage generates an ECL signal. The signal is proportional to the concentration of amyloid beta 1-x present in the plasma. Results for unknowns are reported as the mean concentration of duplicate wells. Data analysis is performed with SoftMax Pro using a 4-parameter logistic fit regression equation. From the results generated the analysis and interpretation is performed as previously described.

What is claimed is:

1. A method of characterizing amyloid beta, the method comprising:
measuring a quantity of amyloid beta in a sample and measuring a quantity of a carboxyl-terminal portion of the amyloid beta available for binding in the sample;
comparing the quantity of the carboxyl-terminal available for binding to the quantity of amyloid beta; and
quantifying, based on the comparison, an amount of oligomeric amyloid beta in the sample.

2. The method of claim 1, wherein
measuring the quantity of amyloid beta includes: contacting the sample with a reporter immunoreagent that binds to a mid-peptide region of the amyloid beta; contacting the sample with an immunoreagent that binds to an amino-terminal region of an amyloid beta peptide; and quantifying a detectable signal from a biomolecular interaction between amyloid beta peptides and the immunoreagents;
measuring the quantity of the carboxyl-terminal portion of amyloid beta available for binding includes: contacting the sample with a second immunoreagent that binds to the carboxyl terminal; and
measuring comprises quantifying a detectable signal from a biomolecular interaction between amyloid beta peptides and the immunoreagents.

3. The method of claim 1, wherein comparing comprises using an algorithm with the measured quantities as inputs, to generate an output comprising a quantified risk of amyloid beta mediated neurodegeneration;
wherein the algorithm comprises: calculating a difference between the quantity of amyloid beta and the quantity of the carboxyl-terminal available for binding; calculating a ratio between the quantity of amyloid beta and the quantity of the carboxyl-terminal available for binding; or calculating a ratio between at least one of (i) the difference or sum between the quantity of amyloid beta and the quantity of the carboxyl-terminal available for binding and (ii) the quantity of amyloid beta.

4. The method of claim 2, wherein the reporter immunoreagent comprises 266, 6E10 or 4G8.

5. The method of claim 2, wherein the reporter immunoreagent is labeled with at least one of: an enzyme; a fluorophore, a chromophore; a chemiluminescent reagent; an affinity tag; or a radioisotopic reagent.

6. The method of claim 2, wherein the detectable signal is generated by an assay selected from the list consisting of colorimetric based Enzyme linked immunosorbent assay (ELISA) enzyme immunoassays (EIA), immunoblot assays, Western blot assays, immunoprecipitation, enzyme linked immunospot, antibody microarray assays, and/or immunohistochemistry assays (IHC), fluoroimmunoassays (FIA), fluorescent bead based immunoassay, flow cytometric assays, fluorescence-activated cell sorting (FACS), radioimmuno assays (RIA), immunocytochemical (ICH) assays, chemiluminescent assays, electrochemilumenescence (ECL) assays, chromatographic assays either by immunaffinity or post column signal generation.

7. The method of claim 1, further comprising:
determining an amount of the monomeric amyloid beta based on the measured quantity of the carboxyl-terminal portion of the amyloid beta available for binding;
determining an amount of the total (monomeric and oligomeric) amyloid beta based on the measured quantity of the amino-terminal portion of the amyloid beta available for binding; and
providing a report that includes an amount of oligomeric amyloid beta in the sample, wherein the amount of oligomeric amyloid beta is the measured quantity of amyloid beta less the determined amount of monomeric amyloid beta.

8. The method of claim 7, wherein quantifying comprises determining a ratio between the amount of monomeric amyloid beta and total amyloid beta in the sample.

9. The method of claim 1, wherein the sample comprises at least one of blood, plasma, serum, or cerebrospinal fluid.

10. The method of claim 2, wherein the immunoreagent specifically binds to Aβ1, Aβ2, Aβ3, Aβ4, Aβ5, Aβ6, Aβ7, Aβ8, Aβ9, Aβ10, or Aβ11.

11. The method of claim 2, wherein the immunoreagent is 3D6, 1E8, MABN639 or AHP1252.

12. The method of claim 2, wherein the second immunoreagent specifically binds to Aβ37, Aβ38, Aβ39, Aβ40, Aβ41, or Aβ42.

13. The method of claim 2, wherein the second immunoreagent is anti-amyloid beta x-40 (2G3, MABN11) or anti-amyloid beta x-42 (MABN12, MABN13 or 05-831-1).

14. The method of claim 1, wherein one or both of the immunoreagents is a reporter antibody or a capture antibody.

15. The method of claim 1, further comprising assessing a subject's susceptibility to developing Alzheimer's disease; determining a stage of Alzheimer's disease; or determining a response to treatment of Alzheimer's disease or determining a treatment regimen.

16. The method of claim 2, further comprising treating the subject with an anti-idiotype antibody prior to contacting with the first and second immunoreagents, the anti-idiotype antibody comprising anti-solanuzumab or anti-bapineuzumab.

17. A kit for characterizing amyloid beta in a sample, the kit comprising:
an antibody specific for a carboxyl terminal epitope of amyloid beta,
an antibody specific for an amino terminal epitope of amyloid beta, and
an antibody specific for a central epitope of amyloid beta.

18. The kit of claim 17, wherein the carboxyl terminal antibody is end-specific for at least one of Aβ37, Aβ38, Aβ39, Aβ40, Aβ41, or Aβ42; and wherein the amino terminal antibody is end-specific for at least one of Aβ1, Aβ2, Aβ3, Aβ4, Aβ5, Aβ6, Aβ7, Aβ8, Aβ9 or Aβ10.

19. A method of characterizing amyloid beta, the method comprising:
measuring a quantity of amyloid beta in a sample and measuring a quantity of a carboxyl-terminal portion of the amyloid beta available for binding in the sample;
assessing whether the quantity of the carboxyl-terminal available for binding meets a dilutional linearity acceptance criterion; and
determining, based on the assessment, a relative amount of monomeric amyloid beta versus oligomeric amyloid beta in the sample.

20. The method of claim 19, wherein the carboxyl-terminal quantity meeting the dilutional linearity acceptance criterion indicates the sample contains mostly monomeric amyloid beta; and wherein the carboxyl-terminal quantity failing to meet the dilutional linearity acceptance criterion indicates the sample contains oligomeric amyloid beta.
